(19) United States
(12) Patent Application Publication
(10) Pub. No.: US 2010/0324693 A1
(43) Pub. Date: Dec. 23, 2010

(54) CARPOMETACARPAL JOINT SPACER

(76) Inventor: Frederick H. HARDENBROOK, Egg Harbor Township, NJ (US)

Correspondence Address: CARTER, DELUCA, FARRELL & SCHMIDT, LLP, 445 BROAD HOLLOW ROAD, SUITE 420, MELVILLE, NY, 11747, US

(21) Appl. No.: 12/821,911

(22) Filed: Jun. 23, 2010

Related U.S. Application Data
(60) Provisional application No. 61/219,713, filed on Jun. 23, 2009.

(51) Int. Cl. A61F 2/42

(52) U.S. Cl. 623/21.11

(57) ABSTRACT

A swellable, resilient, flexible carpometacarpal (CMC) joint spacer is provided that is made of a swellable fluid absorbing polymeric medium, the spacer being dimensioned and configured to fit into the space between a natural or replacement trapezium and adjacent metacarpal bones and restore separation therebetween. In embodiments, the spacer has a cylindrical shape. In embodiments, the polymeric medium is a hydrogel. In embodiments, the spacer contains an internal reinforcement member. In embodiments, the spacer is delivered to a target site in at least a partially dehydrated state, whereupon it hydrates and expands to create positive pressure against opposing surfaces, thereby causing separation of the joint.

### CROSS-REFERENCE TO RELATED APPLICATION

This application claims priority of U.S. Provisional Application Ser. No. 61/219,713, filed Jun. 23, 2009 and is incorporated herein by reference in its entirety.

### BACKGROUND

1. Field of the Invention

The present disclosure is directed to prosthetic implants for distracting or maintaining separation of joints.

2. Description of Related Art

The basal joint of the thumb, the carpometacarpal (CMC) joint, is one of the joints most commonly affected by arthritis. Until recently typical surgical treatment for this condition involved a large incision, excision of the entire trapezium and interposition of a tendon harvested from the forearm. Total trapezium replacements or trapezial implants made of materials such as silicone, titanium, zirconia, and pyrocarbon have been developed but with potential side effects, including conditions such as silicone sinovitis and subluxation of the joint. Recently resorbable spacers have been used with an arthroscopic approach to preserve the joint capsule, the Graft Jacket by Wright Medical and the Artelon marketed by Small Bone Innovations. However both potentially have adverse responses in the body during resorption. There exists a need for a highly biocompatible device that can be inserted minimally invasively via arthroscopic approach that will restore joint separation.

### SUMMARY

A swellable, resilient, flexible carpometacarpal (CMC) joint spacer is provided that is made of a swellable fluid absorbing polymeric medium, the spacer being dimensioned and configured to fit into the space between a natural or replacement trapezium and adjacent metacarpal bones and restore separation therebetween. In embodiments, the spacer has a cylindrical shape. In embodiments, the polymeric medium is a hydrogel. In embodiments, the spacer contains an internal reinforcement member. In embodiments, the internal reinforcement member is dimensioned and configured to receive and support a fastener for affixing the spacer to surrounding hard or soft tissue. In embodiments, the internal reinforcement member is pre-attached to a fastener which extends outwardly from the spacer, the fastener adapted and configured for attaching to surrounding hard or soft tissue for purposes of stabilizing the position of the spacer in the CMC cavity. In embodiments, the spacer is delivered to a target site in at least a partially dehydrated state, whereupon it hydrates and expands to create positive pressure against opposing surfaces, thereby causing separation of the joint.

### BRIEF DESCRIPTION OF THE DRAWINGS

FIG. 1 is a dorsal view of a human skeletal hand.

FIG. 2 is a perspective view of a hydrated carpometacarpal joint spacer according to the present disclosure.

FIG. 3 is a perspective view of a dehydrated carpometacarpal joint spacer according to the present disclosure.

FIG. 4 is a perspective view of an embodiment of a dehydrated carpometacarpal joint spacer in folded configuration according to the present disclosure.

### DETAILED DESCRIPTION OF PREFERRED EMBODIMENTS

A swellable, resilient, flexible carpometacarpal (CMC) joint spacer according to the present disclosure is utilized to restore separation between the trapezium bone and metacarpal bones in a CMC joint, e.g., in a patient suffering from a severe case of osteoarthritis, from trauma or for any other reason requiring replacement or augmentation of the trapezium or adjacent hard or soft tissue. The spacer may be delivered in a hydrated state allowing for immediate visualization of the joint space prior to closing of the incision. Alternatively, the spacer may be at least partially dehydrated, thus providing a reduced dimensional aspect which swells to final dimensions upon hydration. In this manner, a smaller incision may be utilized, thus reducing the amount of surgically induced trauma at the implantation site. The ability to swell to an expanded configuration in situ allows the spacer to partially or substantially fill the required anatomical space and provide sufficient separation to mimic the function of the original physiological architecture. For example, an expanded spacer is dimensioned and configured to provide proper joint spacing. In embodiments, the spacer is provided in an anatomically friendly, cylindrical disc shape to induce proper positioning of the thumb (first) metacarpal bone and reduce chance of joint dislocation due to prosthesis malfunction.

A hydrogel spacer according to the present disclosure provides an improvement over existing implants in that its high biocompatibility will decrease the occurrence of synovitis at the joint due to prosthetic particulates. The anatomic, yet simple, geometry provides excellent positioning post implantation and a potential decrease in the risk of joint subluxation. The flexibility of the spacer allows for the spacer to conform nicely to the natural and/or replacement joint anatomy resulting in a better patient to patient fit of the device. In embodiments, a swellable, resilient spacer according to the present disclosure may be implanted using minimally invasive surgery as a result of the ability of the spacer to achieve an optimum implantable substantially reduced configuration (also referred to herein as the first configuration) and further ability to expand anisotropically or isotropically to an expanded configuration which is adapted and configured to at least partially fill the desired joint space. The spacer may be delivered in a dehydrated flexible state allowing for a minimally invasive arthroscopic surgical approach. The flexibility of the dehydrated spacer allows for rolling prior to insertion thus allowing for smaller incisions and resulting in better retention of the native joint capsule.

A minimally invasive CMC joint spacer according to the present disclosure provides an improvement over conventional surgical approaches by replacing the current use of cadaveric tissue, eliminating the chance for common side effects including silicone sinovitis, and preserving native tissue by replacing a ligament reconstruction tendon interplasty (LRTI) surgical approach. In embodiments, the spacer is delivered in at least a partially dehydrated state in which the length and width of the device are the same as the length and width of the device once hydrated. This will allow the surgeon to better see joint surface coverage once placed. The spacer will swell once placed in the joint space and exposed to bodily fluids. A hydrophilic spacer will swell upon absorption of fluids thus creating positive pressure against opposing surfaces to create the desired joint separation.

In embodiments, the implant has a first configuration and a second configuration. The first configuration is has less volume than the second configuration. In embodiments, the second configuration is achieved by hydration of the spacer and is thicker than the first configuration. The spacer may be delivered in a dehydrated state with a thickness notably smaller than that of the hydrated state of the second configuration. When still flexible due to partial dehydration and/or the presence of plasticizer, the spacer may be compacted by rolling, folding or by providing an accordion configuration. In embodiments, length and width of the spacer may remain constant when going from the dehydrated state to the hydrated state. This allows the surgeon to have proper visualization of joint space coverage without the requirement of waiting for spacer hydration.

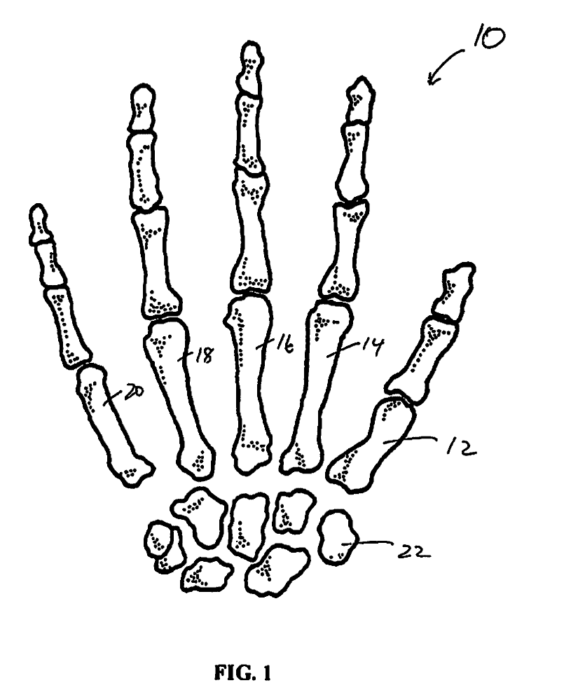

FIG. 1 depicts a dorsal view of a human skeletal left hand 10 which includes a first metatarsal 12 (thumb), a second metatarsal 14, a third metatarsal 16, a fourth metatarsal 18 and a fifth metatarsal, 20. The hand also includes a carpus including a trapezium 22. A spacer, according to the present disclosure, is utilized to buttress the space between the trapezium (natural or artificial) and surrounding hard or soft tissue and is dimensioned and configured to occupy and exert positive pressure the space therebetween.

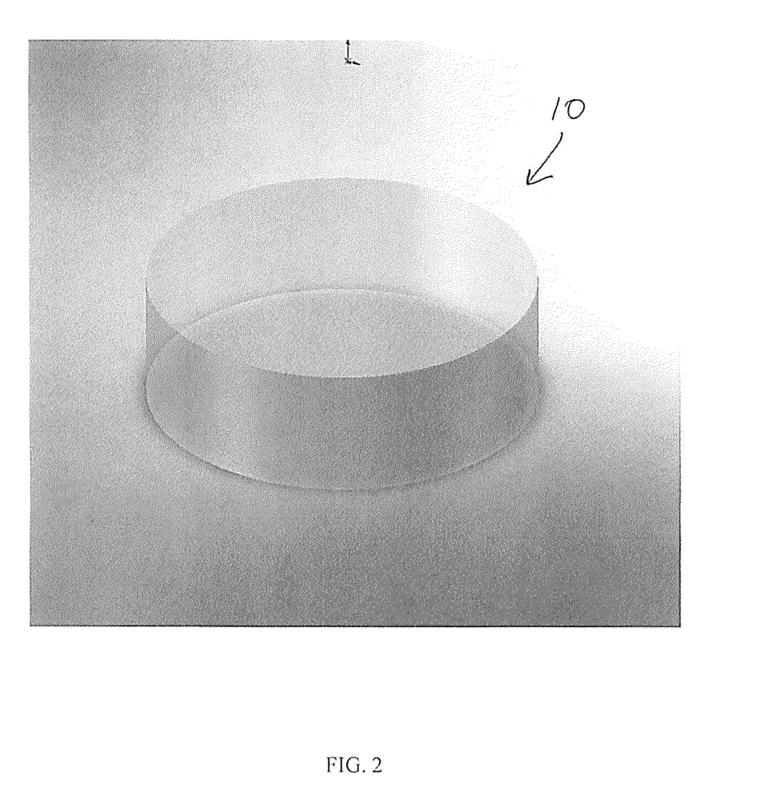

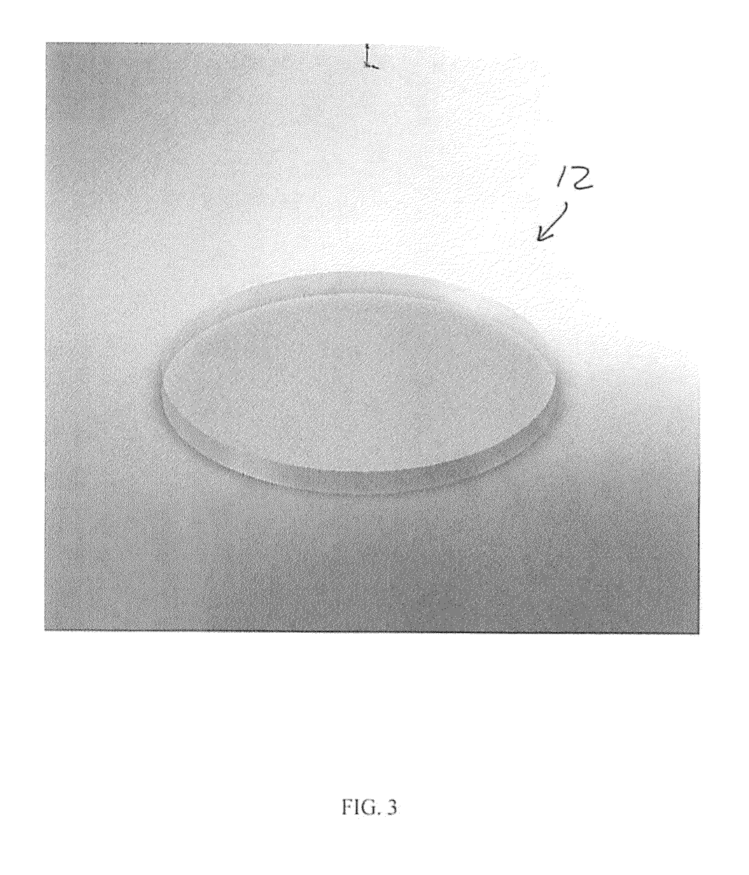

Referring to FIG. 2, an example of a cylindrical spacer 10 is shown in perspective in its hydrated state. FIG. 3 is a perspective view of an example of a cylindrical spacer 12 in a reduced, dehydrated state. In embodiments, an interiorly disposed reinforcement member may be utilized to help prevent creep under loads and/or as a buttress for fixation of a fastener, e.g., for suture fixation, staple fixation, screw fixation, barb fixation and the like. The reinforcement member allows for improved fastener retention by the implant for long term placement.

When fully hydrated, the swellable, flexible spacer is resilient enough to hold its shape despite the rigors and stress of normal hand movements. In embodiments, the surface of the spacer is lubricious which helps prevent wear on the spacer as well as to surrounding bones, tendons and ligaments. In embodiments, the chemical make-up of the swellable polymer can be altered to make the portions of, or the entire spacer, softer or stiffer, i.e., more or less elastic.

Fluid absorbing polymers are well-suited for manufacturing a swellable, resilient spacer in accordance with the present disclosure. Suitable fluid absorbing polymers include synthetic polymers such as poly(ethylene glycol), poly(ethylene oxide), partially or fully hydrolyzed poly(vinyl alcohol), poly(vinylpyrrolidone), poly(ethyloxazoline), poly(ethylene oxide)-co-polypropylene oxide) block copolymers (poloxamers and meroxapols), poloxamines, carboxymethyl cellulose, and hydroxyalkylated celluloses such as hydroxyethyl cellulose and methylhydroxypropyl cellulose, and natural polymers such as polypeptides, polysaccharides or carbohydrates such as Ficoll™, polysucrose, hyaluronic acid, dextran, heparan sulfate, chondroitin sulfate, heparin, or alginate, and proteins such as gelatin, collagen, albumin, or ovalbumin or copolymers or blends thereof. As used herein, “celluloses” includes cellulose and derivatives of the types described above; “dextran” includes dextran and similar derivatives thereof. Examples of materials that can be used to form a hydrogel include modified alginates. Alginate is a carbohydrate polymer isolated from seaweed, which can be crosslinked to form a hydrogel by exposure to a divalent cation such as calcium. Alginate is ionically crosslinked in the presence of divalent cations, in water, at room temperature, to form a hydrogel matrix. Modified alginate derivatives may be synthesized which have an improved ability to form hydrogels.

Additionally, polysaccharides which gel by exposure to monovalent cations, including bacterial polysaccharides, such as gellan gum, and plant polysaccharides, such as carrageenans, may be crosslinked to form a hydrogel using methods analogous to those available for the crosslinking of alginates described above. Polysaccharides which gel in the presence of monovalent cations form hydrogels upon exposure, for example, to a solution comprising physiological levels of sodium. Hydrogel precursor solutions also may be osmotically adjusted with a nonion, such as mannitol, and then injected to form a gel.

Other polymeric hydrogel precursors include polyethylene oxide-polypropylene glycol block copolymers such as Pluronics™ or Tetronics™, which may be crosslinked by hydrogen bonding and/or by a temperature change. Other materials which may be utilized include proteins such as fibrin, collagen and gelatin. Polymer mixtures also may be utilized. For example, a mixture of polyethylene oxide and polyacrylic acid which gels by hydrogen bonding upon mixing may be utilized. In one embodiment, a mixture of a 5% w/w solution of polyacrylic acid with a 5% w/w polyethylene oxide (polyethylene glycol, polyoxyethylene) 100,000 can be combined to form a gel over the course of time, e.g., as quickly as within a few seconds.

Water soluble polymers with charged side groups may be crosslinked by reacting the polymer with an aqueous solution containing ions of the opposite charge, either cations if the polymer has acidic side groups or anions if the polymer has basic side groups. Examples of cations for cross-linking of the polymers with acidic side groups to form a hydrogel are monovalent cations such as sodium, divalent cations such as calcium, and multivalent cations such as copper, calcium, aluminum, magnesium, strontium, barium, and tin, and di-, tri- or tetra-functional organic cations such as alkylammonium salts. Aqueous solutions of the salts of these cations are added to the polymers to form soft, highly swollen hydrogels and membranes. The higher the concentration of cation, or the higher the valence, the greater the degree of cross-linking of the polymer. Additionally, the polymers may be crosslinked enzymatically, e.g., fibrin with thrombin. The polymers can be covalently crosslinked as well through the addition of ethylene diamine, NBS or a host of crosslinking agents routinely to react with amino, nitrile, urethane and carboxylic functional groups found on the polymer chain.

Suitable ionically crosslinkable groups include phenols, amines, imines, amides, carboxylic acids, sulfonic acids and phosphate groups. Negatively charged groups, such as carboxylate, sulfonate and phosphate ions, can be crosslinked with cations such as calcium ions. The crosslinking of alginate with calcium ions is an example of this type of ionic crosslinking. Positively charged groups, such as ammonium ions, can be crosslinked with negatively charged ions such as carboxylate, sulfonate and phosphate ions. Preferably, the negatively charged ions contain more than one carboxylate, sulfonate or phosphate group.

Anions for cross-linking of the polymers to form a hydrogel are monovalent, divalent or trivalent anions such as low molecular weight dicarboxylic acids, for example, terepthalic acid, sulfate ions and carbonate ions. Aqueous solutions of the salts of these anions are added to the polymers to form soft, highly swollen hydrogels, as described with respect to cations.

A variety of polycations can be used to complex and thereby stabilize the polymer hydrogel into a semi-permeable surface. Examples of materials that can be used include polymers having basic reactive groups such as amine or imine groups, having a preferred molecular weight between 3,000 and 100,000, such as polyethylenimine and polylysine. These are commercially available. One polycation is poly(L-lysine); examples of synthetic polyamines are: polyethyleneimine, poly(vinylamine), and poly(allyl amine). There are also natural polycations such as the polysaccharide, chitosan.

In embodiments, the spacer is made of a hydrogel. Prior to coagulation, the liquid form of a suitable hydrogel is used to form the expanded configuration as it would be in the hydrated state. The hydrogel is then coagulated to form the spacer in an expanded configuration. The spacer may then dehydrated to a xerogel state which reduces the volume of the spacer to the reduced configuration. Many hydrogel polymers behave in a similar manner, which is to say they can be deformed, frozen into a deformed shape and they can maintain that shape indefinitely or until, e.g., a temperature change causes the polymer to “relax” into the shape originally held prior to freezing. This property can be referred to as shape memory or frozen deformation by those skilled in the art.

The temperature at which frozen deformation occurs is referred to as the glass transition temperature or T_{g}. At T_{g} several polymer properties such as density, entropy and elasticity may sharply change. Many polymers can be mixed with agents that can have a drastic effect on a polymer T_{g}. Polymers which absorb fluid are of particular interest and water is the preferred T_{g} altering agent. Hydrogels which contain less than about five percent water may be considered dehydrated or xerogels. The T_{g} of a xerogel will change as it absorbs fluids containing water. Once the T_{g} becomes lower than ambient the now partially hydrated hydrogel becomes pliant and may be elastically deformed. If the polymer is held in a state of elastic deformation while the T_{g} is raised above ambient the polymer will maintain the deformed state indefinitely. This can be accomplished by either lowering the ambient temperature (freezing) or by returning the polymer to its xerogel state thus raising the T_{g}.

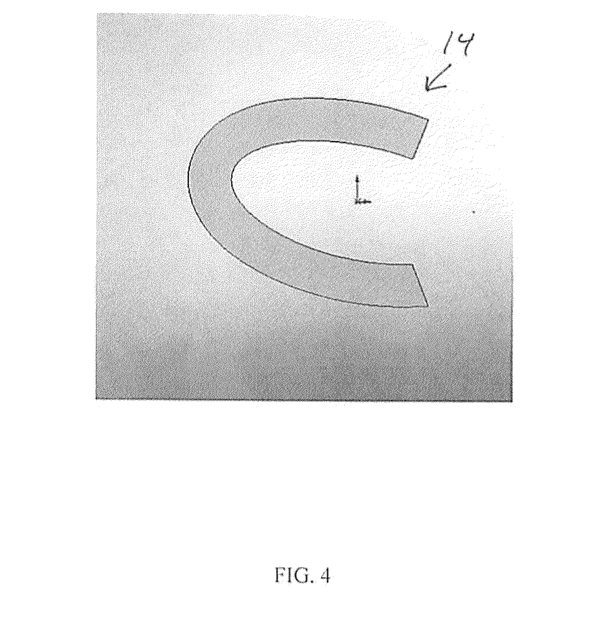

Using this method, hydrogel articles may be produced with vastly differing xerogel shapes compared to hydrated shapes. This is especially useful in cases such as medical implants where, in delivering a spacer into the human body, every care should be taken to reduce trauma to the patient. A spacer which is shaped, e.g., as the spacer shown in FIGS. 2 or 3 can be re-shaped into any compressed shape including, e.g., a tapered elongate rod, folded in a “C” shape, an accordion shape, and the like in order to facilitate minimally invasive implantation. FIG. 4 is a perspective view of a dehydrated spacer in the “C” configuration 14. Alternatively, a portion of the spacer can be compressed as compared to another portion of the spacer. Indeed, various frozen shapes may be utilized to facilitate implantation and situation of the spacer. Once the spacer is indwelling and has absorbed water containing liquids it will substantially return to the expanded shape and maintain that shape indefinitely. In embodiments the spacer has a modulus of elasticity of from about 1 MPa to about 10 MPa. It should be understood that, in embodiments, various shapes may be cast to adjust to a particular patient's anatomy. As used herein, “substantially” is intended to mean any of “approximately”, “nearly” or “precisely.”

A preferred polymer configuration includes two polymer phases of different hydrophilicity, the less hydrophilic phase having higher content of hydrophobic groups and more hydrophilic phase having higher content of hydrophilic groups. The less hydrophilic phase is preferably crystalline and more hydrophilic phase is preferably amorphous, as can be established from X-ray diffraction.

Advantageous hydrophobic groups are pendant nitrile substituents in 1,3 positions on a polymethylene backbone, such as poly(acrylonitrile) or poly(methacrylonitrile). The hydrophilic phase may preferably contain a high concentration of ionic groups. Preferred hydrophilic groups are derivatives of acrylic acid and/or methacrylic acid including salts, acrylamidine, N-substituted acrylamidine, acrylamide and N-substituted acryl amide, as well as various combinations thereof. A particularly preferred combination contains approximately two thirds acrylic acid and its salts (on molar basis), the rest being a combination of plain and N-substituted acrylamides and acrylamidines.

At least one polymeric component is preferably a multiblock copolymer with alternating sequences of hydrophilic and hydrophobic groups. Such sequences are usually capable of separating into two polymer phases and form strong physically crosslinked hydrogels. Such multiblock copolymers can be, for example, products of hydrolysis or aminolysis of polyacrylonitrile or polymethacrylonitrile and copolymers thereof. For convenience, polymers and copolymers having at least about 80 molar % of acrylonitrile and/or methacrylonitrile units in their composition may be referred to as “PAN”. Hydrolysis and aminolysis of PAN and products thereof are described, for example, in U.S. Pat. Nos. 4,107,121; 4,331,783; 4,337,327; 4,369,294; 4,370,451; 4,379,874; 4,420,589; 4,943,618, and 5,252,692, each being incorporated herein by reference in their respective entireties.

A preferred fluid absorbing polymer for the spacer herein is a synthetic composite of a cellular (or domain) type with continuous phase formed by a hydrophobic polymer or a hydrophilic polymer with low to medium water content forming a “closed cell” spongy structure that provides a composite with good strength and shape stability. Examples of suitable polymers are polyurethanes, polyureas, PAN, and highly crystalline multiblock acrylic and methacrylic copolymers. The polymer should be sufficiently permeable to water. More preferably, the continuous phase is formed by a strong hydrophilic polymer with sufficient permeability for water but impermeable to high-molecular solutes. Examples of such polymers are highly crystalline hydrogels based on segmented polyurethanes, polyvinylalcohol or multiblock acrylonitrile copolymers with derivatives of acrylic acid. Typically, suitable polymers for the continuous phase in cellular composites have a water content in fully hydrated state between about 60% by weight and about 90% by weight, preferably between about 70% and about 85% by weight.

The second component of the fluid absorbing polymer may be a highly hydrophilic polymer of high enough molecular weight to prevent permeation of the hydrophilic polymer through the continuous phase. This component is contained inside the matrix of the continuous phase. The entrapped hydrophilic polymers (the so-called “soft block”) may be high-molecular weight water-soluble polymers, associative water-soluble polymers or highly swellable hydrogels containing, in a fully hydrated state, an amount of hydration which is preferably at least about 5% greater than the hydrophobic component. For example, the second component hydrated to at least about 65% when the first component is hydrated to about 60%. In embodiments, e.g., from the second component could be fully hydrated at from about 95% of water and up to about 99.8% of water. Such hydrogels are very weak mechanically. However, it may not matter in composites where such polymers' role is generation of osmotic pressure rather than load-bearing, with e.g., compression strength in full hydration in the range of about 0.01 MN/m² or lower.

A system with closed cells (or domains) containing highly swellable or water-soluble polymers can form composites with very high swelling pressure as needed for the spacer anchoring function. Examples of suitable hydrophilic polymers are high-molecular weight polyacrylamide, polyacrylic acid, polyvinylpyrrolidone, polyethyleneoxide, copolymers of ethyleneoxide and propyleneoxide or hyaluronic acid; covalently crosslinked hydrogels such as hydrophilic esters or amides of polyacrylic or polymethacrylic acids; and physically crosslinked hydrogels, such as hydrolyzates or aminolysates of PAN.

Particularly suitable are associative water-soluble polymers capable of forming very highly viscous solutions or even soft physical gels. Preferred are associative polymers containing negatively charged groups, such as carboxylates, sulpho-groups, phosphate groups or sulfate groups. Particularly preferred are associative polymers formed by hydrolysis and/or aminolysis of PAN to high but finite conversions that leave a certain number of nitrile groups (typically, between about 5 and 25 molar %) unreacted.

Preferred fluid absorbing polymer composites have both a continuous phase and a dispersed phase formed by different products of hydrolysis or aminolysis of PAN. In this case, both components are compatible and their hydrophobic blocks can participate in the same crystalline domains. This improves anchorage of the more hydrophilic component and prevents its extraction or disassociation. The size of more hydrophilic domains may vary widely, from nanometers to millimeters, preferably from tens of nanometers to microns.

The ratio between the continuous discrete phase (i.e., between more hydrophobic and more hydrophilic components may vary from about 1:2 to about 1:100 on a dry weight basis, and a preferred ratio ranges from about 1:5 to about 1:20. Examples of compositions and implants are described in U.S. Pat. Nos. 6,264,695 and 6,726,721, both of which are incorporated herein by reference in their entireties. A preferred method of making the fluid absorbing polymer composite is described in U.S. Pat. No. 6,232,406, herein incorporated by reference in its entirety.

Examples of particularly suitable hydrogel forming copolymers are prepared by a partial alkaline hydrolysis of polyacrylonitrile (“HPAN”) in the presence of sodium thiocyanate (NaSCN). The resulting hydrolysis product is a multi-block acrylic copolymer, containing alternating hydrophilic and hydrophobic blocks. Hydrophilic blocks contain acrylic acid, acrylamidine, and acrylamide. In embodiments, for example, a PAN hydrolysate polymer (referred to herein HPAN I) (46±1% conversion of hydrolysis) having the following composition: acrylonitrile units ˜53-55%, acrylic acid units ˜22-24%, acrylamide units ˜17-19%, acrylamidine units ˜4-6%, as determined by ¹³C NMR, is dissolved in a suitable solvent such as a ˜55% solution of sodium thiocyanate in water to form a viscous solution. The viscous solution is poured into a porous mold having, e.g., a cavity defining the dimensions of the joint spacer. See, e.g., FIGS. 2 and 3. The solution can then be solvent cast, e.g., by solvent exchange (e.g., water for NaSCN). The pores should be sufficiently small as to not permit the polymer to diffuse or leak out of the mold. In another form, the hydrogel used to make the spacer is obtained by reacting an aquagel of PAN, formed by dissolving the polymer in an aqueous solvating solution such as high concentration of sodium thiocyanate. The resulted solution of PAN is thereupon coagulated through addition of a suitable aqueous solvent or water miscible solvent. The coagulum is further reacted in a hydrolyzing basic or acidic medium. The PAN aquagel can then be processed as a thermoplastic and molded to obtain the desired shape. These methods are described in U.S. Pat. No. 4,943,618.

A more rigid fluid absorbing polymer may be another PAN hydrosylate polymer, referred to herein as HPAN II (28±1% conversion of hydrolysis), having the following composition: acrylonitrile units ˜71-73%, acrylic acid units ˜13-15%, acrylamide units ˜10-12%, acrylamidine units ˜2-4%, as determined by ¹³C NMR, disolved in ˜55% NaSCN which can be solvent cast, washed, dried and cut to a suitable shape.

The spacer optionally includes an interiorly embedded reinforcement member. The reinforcement member occupies at least a portion of the interior of the spacer. The reinforcement member can have a planar configuration or it can be one or more rods or beams of relatively rigid material which can extend for a portion of or the entire length of the spacer or it can consist of two or more members. In embodiments, the reinforcement member may be made of a series of individual fibers or ribbons which are arranged in parallel or non-parallel fashion and extend throughout the spacer. In embodiments the reinforcement member can be a fabric or mesh. Woven, non-woven, knitted or braided configurations are suitable. A reinforcement member may be made of a polymeric material which is natural, e.g., cotton, or synthetic, e.g., polyester, polyamide, or other materials such as metal fiber, fiber glass, and carbon fiber. Methods of making shaped objects from these materials and others are well-known to those skilled in the art. Foils or ribbons herein may also be made of metal or polymeric material and are well-known. Thus, the reinforcement member may be constructed from relatively durable materials including, but not limited to, metal foil, plastic foil, metal fibers, polymeric fibers of materials such as polycarbonate, polyethylene, polypropylene, polystyrene, polyethylene terephthalate, polyamide, polyurethane, polyurea, polysulfone, polyvinyl chloride, acrylic and methacrylic polymers, expanded polytetrafluoroethylene (Goretex®), ethylene tetrafluoroethylene, graphite, etc. These materials can be used either alone, or in a composite form in combination with elastomers or hydrogels. Alternatively, the reinforcement member may be exteriorly disposed, e.g., a jacket which surrounds all or part of the spacer. The reinforcement member is designed to provide support for the spacer and to buttress any fastener which may be used to help maintain the spacer in place in the joint. Suitable fasteners include, but are not limited to sutures, screws, staples, barbs and the like. The reinforcement member may be pre-loaded with one or more fasteners or not, e.g., in the situation where a fastener is applied by the surgeon during implantation.

A CMC spacer herein may be manufactured by providing a mold corresponding to the shape of the fully hydrated spacer. A fluid absorbing liquid polymer is added to the mold and is cured or fixed, e.g., by solvent casting, ionic gelation, photo-polymerization and the like, when it solidifies or coagulates. In the case of solvent casting, the mold may be made of material which is impermeable to the fluid absorbing polymer but permeable to water. The mold is placed in a water bath to extract the solvent (e.g., sodium thiocyanate) which causes the polymer to coagulate. The mold may then be opened and any remaining solvent in the spacer is extracted.

It is contemplated that regions of more or less modulus of elasticity and durability may be incorporated into the CMC spacer. For example, it may be desirable to fashion a portion of the spacer from a relatively more rigid fluid absorbing polymer, e.g., the portions which contact bone. If a softer or more elastic zone of fluid absorbing polymer is desired in the central portion of the spacer, a hydrogel such as HPAN I can be added to the portion of the mold defining the central region. It should be understood that any number of zones of varying or the same elasticity may created in this fashion. In addition, different fluid absorbing polymers can be used to create zones with different properties. If desired, an adhesive can be added between adjacent zones to insure bonding or, e.g., in the case of the HPAN polymers, the layers can be made to naturally adhere to one another. Some co-mingling of liquid fluid absorbing polymers at zone interfaces can provide for an advantageous smooth transition between layers and reduce or eliminate the need for an adhesive between layers.

A CMC spacer herein may be manufactured by providing a reinforcement member of desired configuration and placing it in a mold. A fluid absorbing liquid polymer is added to the mold and surrounds the reinforcement member. In one embodiment, a gap, e.g., about 1-3 mm or more, is left between one or more sides of the reinforcement member and the walls of the mold. Fluid absorbing liquid polymer is allowed to fill the gap between the mold and the reinforcement member. When the fluid absorbing polymer is cured or fixed, e.g., by solvent casting, ionic gelation, photo-polymerization and the like, it solidifies and encapsulates the reinforcement member. In the case of solvent casting, the mold may be made of material which is impermeable to the fluid absorbing polymer but permeable to water. The mold is placed in a water bath to extract the solvent (e.g., sodium thiocyanate) which causes the polymer to coagulate. The mold may then be opened and any remaining solvent in the spacer is extracted. If it is desired to leave one or more sides of the spacer open to the reinforcement member, then the desired side(s) of the reinforcement member is placed up against the wall of the mold to prevent formation of a gap for the liquid fluid absorbing polymer to fill.

In embodiments, the fluid absorbing polymer is made to achieve a strong physical bond to the reinforcement member by incorporating an initial treatment of the reinforcement member with a relatively hydrophobic fluid absorbing polymer to create an encapsulating layer of the relatively hydrophobic fluid absorbing polymer. For example, a hydrogel such as HPAN II is applied to the reinforcement member as a 10% solution by weight in a solvent (sodium thiocyanate 55% by weight in water) and then coagulated onto the reinforcement member by solvent exchange with an aqueous solution such as water. As the polymer coagulates, it shrinks volumetrically around the reinforcement member, causing a tight physical bond to the reinforcement member. If desired, the treated reinforcement member is placed in a mold and a relatively more hydrophilic fluid absorbing polymer in the liquid state is added to create a cohesive continuous polymer matrix which surrounds the reinforcement member. For example, a 10% by weight HPAN I in a 55% by weight sodium thiocyanate solution, is added to the mold. The solvent from the HPAN I solution causes the outermost surface of the coagulated HPAN II layer surrounding the braided fibers to dissolve and allow commingling of the HPAN I and HPAN II hydrogel polymers at the surface interface which forms a strong adhesive bond when the HPAN I and commingled hydrogels are coagulated by solvent exchange. It should be understood that the reinforcement member is optional and that a mold may be filled without such a reinforcement member.

Upon completion of the solvent exchange extraction process the spacer may be hydrated to its fullest extent (˜90% equilibrium water content (EWC)). In this fully hydrated state the spacer is readily deformed under modest loads and the hydrogel, e.g., HPAN I or HPAN II, glass transition temperature (T_{g}) is well below room temperature. This is the “relaxed” state of the spacer, the state to which it will return after loading below the critical level. The critical level is the point at which permanent deformation occurs and is further discussed below. In order to provide a reduced configuration (also referred to herein as the first configuration), the spacer may be allowed to dehydrate and enter the xerogel state. A considerable amount of the spacer's volume is lost when in the xerogel state as compared to the hydrated state. Advantageously, the fully hydrated spacer may be deformed into a desirable insertion shape and the temperature of the spacer is lowered below its T_{g} (near freezing point of water). Such a spacer is in a state of “frozen deformation” and it would retain that deformed shape indefinitely. Once the spacer is warmed above its T_{g}, however, the spacer would recover to its original memorized configuration. In embodiments, when the spacer is in the first configuration, it can be made flexible through incorporation of a plasticizer during manufacture. In this manner, the first configuration may take the form of a flat disc which is flexible. The disc can be rolled or folded into a C-shape, a cigar shape, a taco shape, an accordion shape, or folded into any suitable shape which reduces the dimension of the implant to create an optimum profile for insertion through a minimally invasive incision. Examples of plasticizers include glycerol, propylene glycol, small molecular weight polyethylene glycol and the like. In embodiments, 0-45% glycerol in 0.9% saline solution by weight can be incorporated during manufacture of the spacer, e.g., by immersion in the solution after the coagulation step. For example, 15% glycerol in 0.9% saline may be utilized.

The T_{g} of the hydrogel increases with decreasing water content. This characteristic is exploited by simultaneously raising the T_{g} while deforming the spacer into a desired shape. In other words, as the spacer dehydrates it is freezing the position of the polymer chains. To regain the original shape of the spacer, the T_{g} may be lowered by hydration.

In order to obtain a rod-shape having a cross-sectional ellipsoid shape for implantation, e.g., suppository, bullet, tapered cylinder, etc., from, e.g., the configuration shown in FIGS. 2 or 3, reduction in volume deformation is advantageously maintained radially, substantially parallel with the longitudinal axis. This is accomplished by placing the spacer within a radially collapsible member for exerting circumferential compression on an object, e.g., a spacer, contained within the member. Suitable radially collapsible members include, e.g., a flexible sleeve such as a braided sock or tube, a flexible coil, iris diaphragm, collapsible loop, etc. In a preferred embodiment, the radially collapsible member is porous or semipermeable so that water, either as liquid or as vapor, passes through the member. The collapsible member may be made of an elastic material such as rubber or neoprene fabric which has been made porous by any technique known to those skilled in the art, or a woven or non-woven mesh or braid. The collapsible member may also be made of a flexible metal having sufficient porosity to allow water to exit from the spacer. The collapsible member does, however, need to be stiff enough to be able to exert sufficient compressive force when tension is applied to compress the spacer, i.e., it should not be so elastic that it deforms without being able to exert sufficient compressive force.

In operation, the radially collapsible member exerts substantially equilateral circumferential compression on the spacer by substantially uniformly decreasing in diameter while contacting the spacer. The preferred porous nature of the collapsible member allows water from the spacer to escape into the surrounding environment so that the spacer can become dehydrated. In one embodiment, the sleeve radially collapsible member is stretched in length which causes the inner diameter to decrease, thus compressing the spacer, including, e.g., a reinforcement member, into a desired implantation configuration. A more complete description of a suitable radial compression process is described in U.S. application Ser. No. 11/303,767, herein incorporated by reference in its entirety.

The collapsible member is loaded in tension via any tensioning device known to one skilled in the art, e.g., a pneumatic cylinder, a hydraulic cylinder, springs, weights, pulleys, etc. The tension on the collapsible member can be precisely controlled by regulating the pressure within the tensioning device, translating into constant, controlled radial load on the spacer. In the case of a sleeve collapsible member, once the spacer is loaded into the collapsible member and the collapsible member is tensioned, three things occur: the spacer dehydrates, the spacer deforms, and the collapsible member extends. By varying the tension on the collapsible member, the length of the spacer can optionally be extended, thereby decreasing the minor axis and height. This can also be controlled, to some extent, by the speed of dehydration (temperature, pressure and humidity), with longer dehydration time producing longer spacer length and vise versa. In certain embodiments, one portion of the collapsible member is made to collapse further than other portions to define a spacer having one end which is relatively more compressed than the other end.

Two concerns with respect to drying time and collapsible member tension should be considered. The first is creep, which may set in if the dehydration time is extended unreasonably long (over several days). The second is permanent deformation which may occur if excessive stress is applied to the spacer. Both of these concerns only occur at critical point extremes which are to be avoided. Permanent deformation may occur in the hydrogel spacer if the soft-block domains of the polymer are displaced to a point where they cannot reorient themselves into the original lattice configuration, i.e., the memorized shape. This can happen, e.g., by either deforming the original shape so severely that many of the bonds which hold the soft-blocks in place are severed, or by heating the spacer sufficiently above the T_{g} to cause the soft-block domains to permanently or irrevocably assume a new configuration outside of the originally contemplated structure, which causes an undesirable change in shape. Thus, the melting point of the soft block should not be exceeded. The melting point of the soft block may vary based on the amount of water content. Such melting points may be determined by conventional techniques known to those skilled in the art. For example, at 18% hydration of HPAN I, permanent deformation is manifest at temperatures over 105° C.

In embodiments, the majority of the dehydration process can occur at room temperature over an extended period of time (e.g., 18 to 36 hours). The spacer can be monitored to determine the extent of dehydration and the time period adjusted accordingly. Relative humidity, air circulation, air pressure and room temperature should be controlled during this period. Especially preferred conditions are about 21° C. at 50% relative humidity under moderate airflow. Once the spacer has reached <˜30% water content it may be forced dry at elevated temperature , e.g., from about 25° C. to about 105° C. for typically less than about 24 hours to rapidly remove remaining water. As above, the state of dehydration may be monitored to determine if greater or lesser amounts of time are needed. When the spacer is substantially completely dehydrated, the spacer is fairly rigid in its state of frozen deformation. Alternatively, a slight degree of hydration provides some flexibility to the spacer. The less dehydrated, the more flexible. As stated above, in embodiments, it may be desirable to maintain some flexibility of the spacer to allow the surgeon to manipulate the spacer into different configurations while still allowing the spacer to be placed in the joint space to allow estimation of the final hydrated dimensions. It is contemplated herein that “substantially dehydrated” preferably encompasses from about 12% or less, to about 30% water by weight of the spacer.

Upon completion of forced dehydration, the spacer is extremely stable in terms of shelf life, providing that it is kept dry. Even brief exposure to humidity during the sterilization process should not have significant effects. Temperatures above about 80° C. should be avoided for extended periods as this may bring the spacer above its T_{g} if it has absorbed some small amount of water vapor.

Surface irregularities may be present on a dehydrated compressed spacer which was compressed as described above by a radially collapsible member by virtue, e.g., of some extrusion of the hydrogel through pores or through interstitial spaces of the member. For example, a woven or non-woven collapsible sleeve may have interstitial spaces that allow hydrogel to extrude therein under compressive force. In addition, after radial compression, as described above, the dimensions of the spacer may be different than the ultimate dimensions desired by the practitioner. Both of these instances can be remedied by post-compression thermoforming of the spacer. In this aspect, a dehydrated, compressed spacer is placed within a mold which may be advantageously pre-heated to about 70-150° C., but more preferably, closer to the melting point of the polymer, e.g., about 105° C. Care must be taken to avoid subjecting the spacer to excess heat which causes the hydrogel to exceed its critical point, and thus causing permanent deformation of the spacer. If the temperature is high, the spacer must be quickly removed from the mold to avoid permanent deformation. The mold is machined to the exact desired final dimensions of the xerogel spacer and essentially irons out surface roughness to a substantially smooth surface, which is less abrasive to surrounding tissue when implanted. If desired, and if the xerogel spacer is compressed by a radially compressive member or by gas compression, but has not achieved, e.g., an ideal enough straight rod-like configuration, or if the ends are not sufficiently blunted or otherwise tapered, post-compression thermoforming may be utilized to fine tune the shape as well as remove any surface irregularities which may be present. Post-compression thermoforming may also be utilized to bend a spacer to a desired configuration, e.g., to a boomerang shape.

A spacer according to the disclosure herein may contain a medicinal agent. “Medicinal agent” is used in its broadest sense and it includes any substance or mixture of substances which may have any clinical use. It is to be understood that medicinal agent encompasses any drug, including hormones, antibodies, therapeutic peptides, etc., or a diagnostic agent such as a releasable dye which has no biological activity per se. Thus, in its broadest aspect, a method of delivery herein may be defined as the release of any substance for clinical use, which may or may not exhibit biological activity.

Examples of medicinal agents that can be used include anticancer agents, analgesics, anesthetics, anti-inflammatory agents, growth factors such as BMPs, antimicrobials, and radiopaque materials. Such medicinal agents are well-known to those skilled in the art. The medicinal agents may be in the form of dry substance in aqueous solution, in alcoholic solution or particles, microcrystals, microspheres or liposomes. An extensive recitation of various medicinal agents is disclosed in Goodman and Gilman, The Pharmacological Basis of Therapeutics, 10th ed. 2001, or Remington, The Science and Practice of Pharmacy, 21 ed. (2005). As used herein, the term “antimicrobial” is meant to encompass any pharmaceutically acceptable agent which is substantially toxic to a pathogen. Accordingly, “antimicrobial” includes antiseptics, antibacterials, antibiotics, antivirals, antifungals and the like. Radiopaque materials include releasable and non-releasable agents which render the spacer visible in any known imaging technique such as X-ray radiographs, magnetic resonance imaging, computer assisted tomography and the like. The radiopaque material may be any conventional radiopaque material known in the art for allowing radiographic visualization of a spacer, and may be, e.g., metal wire or flakes made from a biocompatible material, such as titanium, tantalum, stainless steel, or nitinol; or metallic salts (such as barium compounds).

Medicinal agents may be incorporated into the spacer at various points in the manufacturing process. For example, a suitable medicinal agent can be mixed with a fluid absorbing liquid polymer before it is cured or fixed. Alternatively, a suitable medicinal agent may be dissolved into a solvent cast solution and then diffused into the hydrogel in accordance with normal kinetic principles. If the spacer is then dehydrated, the medicinal agent collects in the interstices of the hydrogel.

A spacer according to the disclosure herein may be sterilized by any suitable conventional means, e.g., ethylene oxide, irradiation, etc. and packaged for distribution. A kit containing the sterilized spacer and a package insert describing the spacer along with instructions is useful for medical practitioners.

Techniques for implanting a spacer in hands are well-known. In embodiments, it is contemplated that the spacer may be implanted in the fully hydrated state to allow visualization of the spacer in the target void in the joint. In the present case, minimally invasive implantation techniques are improved and facilitated by the reduced dimension and overall configuration of the reduced or first configuration. In addition, the ability to provide custom implantation shapes allows an optimal insertion shape to be manufactured. For implantation of the spacer into a hand, access to the CMC may, e.g., be performed dorsally. Indeed, palmar, dorsal, medial, and lateral surgical approaches can all be performed. The spacer is designed to reduce tissue trauma due to surgery by allowing implantation of the spacer in a reduced configuration, thus allowing for minimally invasive surgical techniques. The ability to assume a state of reduced configuration advantageously minimizes undue disruption to soft tissue.

In practice, the bony segments are exposed, care being taken to preserve as much as possible soft tissue connections. In embodiments, the unconstricted volume of the spacer, when expanded (also referred to herein as the second configuration), is slightly greater than the space in the joint such that, in situ, the spacer is compressed when situated in the joint. In this manner, the swellable spacer exerts positive pressure against opposing surfaces of the joint, thus maintaining pressure and separation as the hydrophilic spacer is biased to absorb fluid. In embodiments, the surface of the spacer may be shaped to encourage frictional affixation and/or treated to encourage bone ongrowth or ingrowth, as by providing, e.g., surface with barbs, spurs or spikes and/or porous surfaces, by applying to the spacer a growth factor, cell adhesion promoter such as collagen or the like, etc.

A fastener, such as a suture may be used to position and secure the spacer in place. The reinforcement member may be used as a buttress or pledget to secure the fastener to the spacer. As mentioned above, the reinforcement member may be preloaded with the fastener, e.g., a suture, extending out of the spacer which is then used to anchor the spacer to surrounding bone or tissue. When the reinforcement member is not preloaded, a surgeon may pass a fastener such as a suture through any portion of the outside face of the spacer and through the reinforcement member, e.g., a mesh or around a bar or beam, enclosed in the spacer. The intended anatomy for the sutures is simply the surrounding tissues and ligaments. If possible, the fastener(s) could be used to repair and close the joint capsule but if not possible simply suturing the spacer to the surrounding tissue in such a way as to not limit function.

It should be understood that the examples and embodiments provided herein are preferred embodiments. Various modifications may be made to these examples and embodiments without departing from the scope of the disclosure which is defined by any appended claims. For example, those skilled in the art may envision additional polymers and/or hydrogels which can be compacted and shaped according to the techniques described herein. Similarly, the shapes of the hydrated or expanded spacer described herein are exemplary and any suitable expanded spacer shape can be subjected to the techniques described herein to create an optimally shaped, substantially dehydrated spacer for minimally invasive insertion into the implantation space. Although especially well-suited for implantation in a dehydrated state, the spacer can be delivered in a partially or fully hydrated state at the time of implantation. Moreover, those skilled in the art can envision additional radially collapsible members for exerting substantially uniform radial compression on the spacer which are not set forth herein. In addition, process parameters such as temperature, humidity, pressure, time and concentration may be varied according to conventional techniques by those skilled in the art to optimize results. These are merely examples of many modifications those skilled in the art may make.

What is claimed is:

1. A swellable, resilient, flexible carpometacarpal joint spacer comprising a swellable fluid absorbing polymeric medium, the spacer being dimensioned and configured to fit into the space between a natural or replacement trapezium and an adjacent metacarpal bone, the joint spacer having first and second configurations, the first configuration occurring when the joint spacer is at least partially dehydrated, the first configuration being smaller than the second configuration, the second configuration occurring when the joint spacer is at least partially hydrated, wherein the second configuration is dimensioned and configured to exert pressure to separate the trapezium from the adjacent metacarpal bone.

2. The carpometacarpal joint spacer according to claim 1 wherein the swellable fluid absorbing polymer is a hydrogel.

3. The carpometacarpal joint spacer according to claim 2 wherein the joint spacer has a lubricious surface when the hydrogel is at least partially hydrated.

4. The carpometacarpal joint spacer according to claim 1 further comprising an internal reinforcement member.

5. The carpometacarpal joint spacer according to claim 4 wherein the reinforcement member is adapted to receive a fastener for affixing the prosthesis to surrounding hard or soft tissue.

6. The carpometacarpal joint spacer according to claim 4 wherein the reinforcement member has a fastener attached thereto, the fastener extending out of the prosthesis, the fastener adapted to fasten the prosthesis to surrounding hard or soft tissue.

7. The carpometacarpal joint spacer according to claim 1 having a modulus of elasticity of from about 1 MPa to about 10 MPa.

8. The carpometacarpal joint spacer according to claim 1 wherein the second configuration is cylindrical.

9. The carpometacarpal joint spacer according to claim 1 wherein the first configuration is flexible.

10. The carpometacarpal joint spacer according to claim 1 wherein the first configuration is in the shape of a flat disc.

11. The carpometacarpal joint spacer according to claim 9 wherein the first configuration has a C-shaped profile.

12. The carpometacarpal joint spacer according to claim 10 wherein the flat disc is flexible.

13. The carpometacarpal joint spacer according to claim 12 wherein the flat disc is folded into a C-shape, a taco shape, a cigar shape or an accordion shape.

14. The carpometacarpal joint spacer according to claim 1 further comprising a medicinal agent.

15. The carpometacarpal joint spacer according to claim 1 further comprising a plasticizer.

16. A method of treating a joint space between a trapezium and a metacarpal bone comprising making an incision to access the space between a trapezium and a metacarpal bone and inserting into the space a swellable, resilient, flexible carpometacarpal joint spacer made of a swellable fluid absorbing polymeric medium, the spacer being dimensioned and configured to fit into the space between the trapezium and the metacarpal bone, the joint spacer having first and second configurations, the first configuration occurring when the joint spacer is at least partially dehydrated, the first configuration being smaller than the second configuration, the second configuration occurring when the joint spacer is at least partially hydrated, wherein the second configuration is dimensioned and configured to exert pressure to separate the trapezium from the adjacent metacarpal bone, and closing the incision.

17. The method of treating a joint space between a trapezium and a metacarpal bone according to claim 15 wherein the joint spacer is inserted when in the first configuration, whereupon the joint spacer hydrates and expands to create positive pressure against opposing surfaces, thereby causing enlargement of the joint space.
